(19) United States
(12) Patent Application Publication
(10) Pub. No.: US 2013/0030311 A1
(43) Pub. Date: Jan. 31, 2013

(54) BLOOD PRESSURE MEASUREMENT DEVICE

(71) Applicants: Yukiya Sawanoi, Nara-shi (JP); Shingo Yamashita, Kyoto-shi (JP); Toshiaki Yuasa, Moriyama-shi (JP)

(72) Inventors: Yukiya Sawanoi, Nara-shi (JP); Shingo Yamashita, Kyoto-shi (JP); Toshiaki Yuasa, Moriyama-shi (JP)

(73) Assignee: OMRON HEALTHCARE Co., Ltd., Kyoto (JP)

(21) Appl. No.: 13/632,805

(22) Filed: Oct. 1, 2012

Related U.S. Application Data
(63) Continuation of application No. PCT/JP2011/052763, filed on Feb. 9, 2011.

(30) Foreign Application Priority Data
Mar. 30, 2010 (JP) 2010-077971

(51) Int. Cl. A61B 5/022

(52) U.S. Cl. 600/499

(57) ABSTRACT

A blood pressure measurement device compresses a measurement site by a cuff being wrapped around the measurement site. A first fluid bag is provided inside an outer package of the cuff. An indicating member is provided in the cuff so as to contact the outer package and the first fluid bag. The properties of the indicating member change in a visible manner in accordance with a magnitude of a force with which the indicating member is pressed by the outer package and the first fluid bag.

### TECHNICAL FIELD

The present invention relates to a blood pressure measurement device, and more particularly to a blood pressure measurement device for wrapping a cuff around and compressing a measurement site when measuring blood pressure.

### BACKGROUND ART

Blood pressure is one of the indexes used to analyze circulatory diseases. Risk analysis based on blood pressure is effective for preventing cardiovascular diseases such as stroke, heart failure and myocardial infarction. Conventionally, diagnosis has been made using blood pressure measured at a medical institution during a hospital visit, a health checkup, or the like. However, recent studies have shown that blood pressure measured at home can be more helpful in diagnosing circulatory diseases than blood pressure measured at a medical institution. In view of this, blood pressure monitors for home use have become widespread, and over 30 million are found in homes across Japan.

Many blood pressure monitors for home use employ an oscillometric method or a microphone method as the method for measuring blood pressure.

With the oscillometric method, blood pressure is measured as follows. A cuff is wrapped around a measurement site such as an upper arm, and the internal pressure of the cuff (cuff pressure) is increased to exceed the systolic blood pressure by a predetermined pressure (e.g. 30 mmHg). Thereafter, the cuff pressure is released in a gradual or stepwise manner, and a volumetric change in the artery during the process of releasing the cuff pressure is detected as the pressure change (pressure pulse wave oscillation) superimposed on the cuff pressure. The systolic blood pressure and the diastolic blood pressure are determined from the change in this pressure pulse wave oscillation. With the oscillometric method, blood pressure can also be measured by detecting the pressure pulse wave oscillation that arises while increasing the cuff pressure.

On the other hand, with the microphone method, a cuff is wrapped around a measurement site such as an upper arm, and the cuff pressure is increased to exceed the systolic blood pressure by a predetermined pressure, as with the oscillometric method. Thereafter, in the process of gradually releasing the cuff pressure, Korotkoff sounds arising from the artery are detected by a microphone provided in the cuff. The cuff pressure at which the Korotkoff sounds arise is determined as the systolic blood pressure, and the cuff pressure at which the Korotkoff sounds diminish or disappear is determined as the diastolic blood pressure.

In order to accurately measure blood pressure using the above methods for measuring blood pressure, it is necessary to appropriately wrap the cuff around a measurement site such as an upper arm. However, with conventional blood pressure measurement devices, it is difficult to judge whether or not the cuff is appropriately attached, and how the cuff is attached varies depending on the person. This may cause inaccurate blood pressure measurement.

In view of the above, for example, Patent Literature 1 (JP 2005-305028A), Patent Literature 2 (JP 02-114934A) and Patent Literature 3 (JP 2008-188197A) disclose a technique for determining whether or not the wrapping strength of the cuff is appropriate when starting the measurement of blood pressure. On the other hand, Patent Literature 4 (JP 2007-275483A) discloses, as a technique for supporting correct attachment of the cuff to a measurement site, a technique whereby the cuff is provided with a member for positioning an air bladder in the cuff in relation to the upper arm.

Patent Literature 1: JP 2005-305028A

Patent Literature 2: JP 02-114934A

Patent Literature 3: JP 2008-188197A

Patent Literature 4: JP 2007-275483A

### SUMMARY OF INVENTION

However, according to the techniques described in Patent Literature 1 to Patent Literature 3, the wrapping strength of the cuff is determined in the process of wrapping the cuff around the measurement site and increasing the cuff pressure, namely when starting the measurement of blood pressure. Therefore, this technique does not allow the wrapping strength to be determined before the measurement of blood pressure is started, namely before wrapping of the cuff on the person being measured is completed. For this reason, with this technique, when the wrapping strength of the cuff has been determined to be inappropriate, it is necessary to stop the measurement, re-wrap the cuff, and then start the measurement again.

Furthermore, although the technique described in Patent Literature 4 provides a mark indicating the attachment direction of the cuff and therefore makes it easy for the person being measured to recognize the attachment direction of the cuff, it does not allow the person to determine whether or not the cuff has been attached correctly.

Therefore, one or more embodiments of the present invention allows a person being measured to recognize whether or not the cuff has been attached correctly, before blood pressure is measured using a blood pressure measurement device.

A blood pressure measurement device according to one or more embodiments of the present invention is provided with a cuff that compresses a measurement site by being wrapped therearound. The blood pressure measurement device includes an outer package that covers an outside of the cuff, a first fluid bag provided inside the outer package, and an indicating member provided in the cuff so as to contact the outer package and the first fluid bag. The properties of the indicating member change in a visible manner in accordance with a magnitude of a force with which the indicating member is pressed by the outer package and the first fluid bag.

According to one or more embodiments of the present invention, the indicating member includes a second fluid bag in which a plurality of fluids are enclosed, the plurality of fluids changing in volume by different amounts when pressed with a predetermined force.

According to one or more embodiments of the present invention, the indicating member further includes a display unit that displays the change in the properties, the display unit is provided with an indicator that shows a magnitude of a force with which the second fluid bag is pressed by the outer package and the first fluid bag, and the indicator is marked on the display unit and has a shape whose center does not coincide with a center of the display unit.

According to one or more embodiments of the present invention, the indicating member is provided in plurality, and the plurality of indicating members is arranged in a direction that intersects a wrapping direction in which the cuff is wrapped around the measurement site.

According to one or more embodiments of the present invention, the indicating member is provided in plurality, and the plurality of indicating members is arranged in a wrapping direction in which the cuff is wrapped around the measurement site.

According to one or more embodiments of the present invention, the pressure measurement device further includes an indicator that shows a magnitude of a force with which the indicating member is pressed by the outer package and the first fluid bag.

One or more embodiments of the present invention allow the person being measured to, when attaching the cuff, visually check the wrapping strength of the cuff by visually checking a change in the properties of the indicating member. In this way, the person being measured can recognize whether or not the cuff has been attached correctly, before measuring blood pressure.

### BRIEF DESCRIPTION OF DRAWINGS

FIG. 1 is a perspective diagram showing an external view of a blood pressure monitor, which is one embodiment of a blood pressure measurement device of the present invention.

FIG. 2 is a functional block diagram showing a configuration of the blood pressure monitor shown in FIG. 1.

FIG. 3 shows a cuff of the blood pressure monitor shown in FIG. 1 in an opened state.

FIG. 4 is a cross-sectional diagram of the cuff taken along the IV-IV line shown in FIG. 3.

FIG. 5 shows the state where the cuff of the blood pressure monitor shown in FIG. 1 has been wrapped around a measurement site and fixed in place.

FIG. 6 shows a cross-sectional configuration of the cuff shown in FIG. 5 in the vicinity of a window of a wrapping strength indicating unit.

FIG. 7 shows a fluid bag of FIG. 6 as viewed through the window from the direction of arrow A1.

FIG. 8 is a cross-sectional diagram of the vicinity of the window of the wrapping strength indicating unit when the wrapping strength has been increased from the state shown in FIG. 6.

FIG. 9 shows a fluid bag of FIG. 8 as viewed through the window from the direction of arrow A1.

FIG. 10 is a diagram for explaining a change in the position of a boundary line in the window of the cuff of the blood pressure monitor shown in FIG. 1.

FIG. 11 is a diagram for explaining a change in the position of a boundary line in the window of the cuff of the blood pressure monitor shown in FIG. 1.

FIG. 12 is a diagram for explaining a change in the position of a boundary line in the window of the cuff of the blood pressure monitor shown in FIG. 1.

FIG. 13 shows a modification of an indicator on the window of the cuff of the blood pressure monitor shown in FIG. 1.

FIG. 14 shows a modification of arrangement of wrapping strength indicating units provided on the cuff of the blood pressure monitor shown in FIG. 1.

FIG. 15 shows a modification of arrangement of wrapping strength indicating units provided on the cuff of the blood pressure monitor shown in FIG. 1.

FIG. 16 shows another modification of arrangement of wrapping strength indicating units provided on the cuff of the blood pressure monitor shown in FIG. 1.

FIG. 17 shows another modification of arrangement of wrapping strength indicating units provided on the cuff of the blood pressure monitor shown in FIG. 1.

FIG. 18 shows yet another modification of arrangement of wrapping strength indicating units provided on the cuff of the blood pressure monitor shown in FIG. 1.

FIG. 19 is a flowchart of processing for measuring blood pressure executed by the blood pressure monitor shown in FIG. 1.

### DETAILED DESCRIPTION OF INVENTION

A description is now given of an embodiment of a blood pressure measurement device of the present invention with reference to the drawings. In the following description, the same components and constituent elements are given the same reference signs, and their names and functions are not repeatedly explained.

### 1. External Configuration of Blood Pressure Monitor

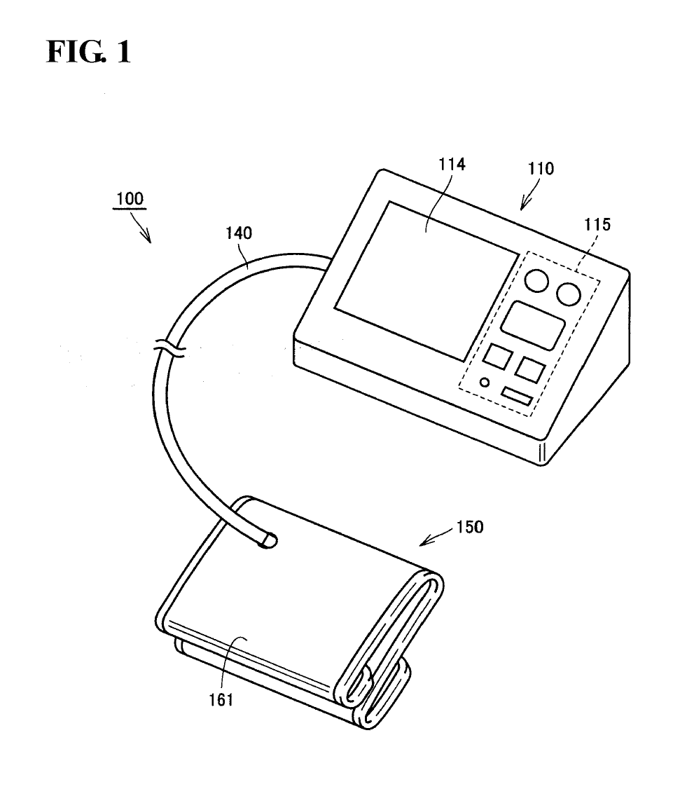

FIG. 1 is a perspective diagram showing an external view of a blood pressure monitor, which is one embodiment of a blood pressure measurement device of the present invention.

Referring to FIG. 1, a blood pressure monitor 100 is mainly provided with a device main body 110 and a cuff 150. The device main body 110 includes a display unit 114 and an operation unit 115. The display unit 114 displays results of the measurement of blood pressure, pulse rate and the like in a visible manner using numerical values, graphs, and so on. For example, a liquid crystal panel may be used as the display unit 114. A power button, a measurement start button, and the like are arranged on the operation unit 115.

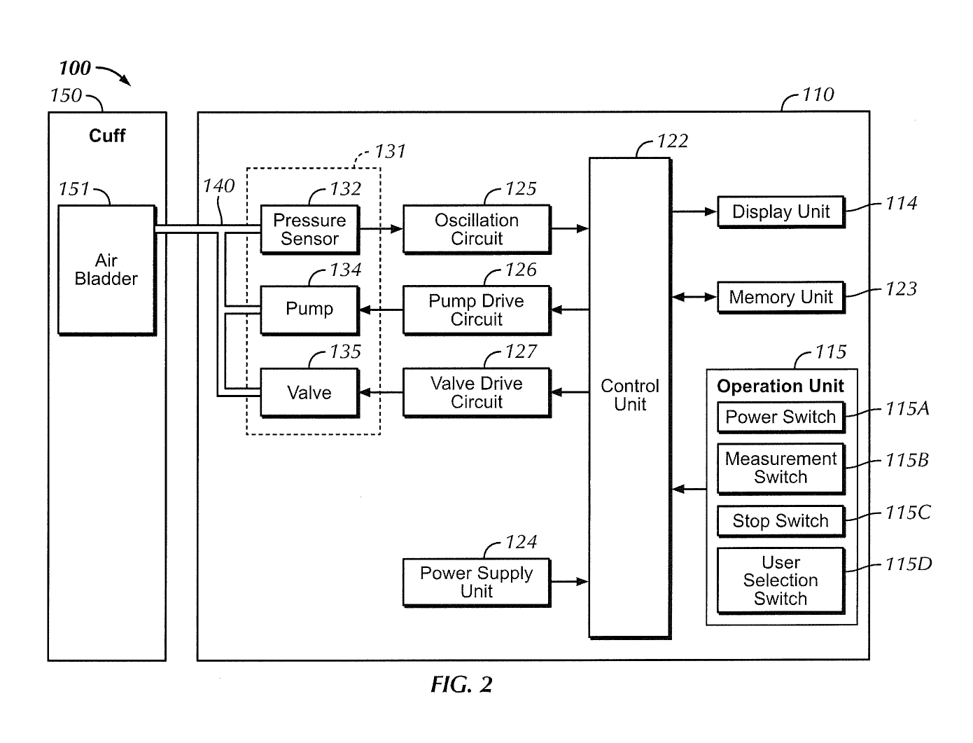

The cuff 150 is intended to be wrapped around a measurement site of the person being measured, and has a belt-like outer shape. The cuff 150 has an air bladder 151 (see FIGS. 2 to 4) and a bag-like cover body 161. The air bladder 151 serves as a fluid bag for compressing the measurement site. The bag-like cover body 161 serves as an outer package for wrapping the air bladder 151 around the measurement site and fixing the wrapped air bladder 151 in place. The air bladder 151 is housed in a space provided inside the bag-like cover body 161.

The cuff 150 and the device main body 110 are connected by an air tube 140 serving as a connecting tube. The air tube 140 is made of a flexible tube. One end thereof is connected to an air system component 131 for measuring blood pressure (see FIG. 2), which is provided in the device main body 110, and the other end thereof is connected to the aforementioned air bladder 151 of the cuff 150. The air system component 131 will be described later.

### 2. Block Configuration of Blood Pressure Monitor

FIG. 2 is a functional block diagram showing the configuration of the blood pressure monitor 100.

Referring to FIG. 2, the air system component 131 for measuring blood pressure is provided in the device main body 110 of the blood pressure monitor 100. The air system component 131 inflates or deflates the air bladder 151 contained in the cuff 150 via the air tube 140. The air system component 131 for measuring blood pressure includes a pressure sensor 132 that detects the pressure inside the air bladder 151, and a pump 134 and a valve 135 for inflating and deflating the air bladder 151. Furthermore, an oscillation circuit 125, a pump drive circuit 126 and a valve drive circuit 127 are also provided in the device main body 110 in association with the air system component 131 for measuring blood pressure.

The device main body 110 is also provided with a control unit 122, a memory unit 123, the display unit 114, the operation unit 115, and a power supply unit 124. The control unit 122 centrally controls and monitors the components. The memory unit 123 stores therein programs for causing the control unit 122 to execute predetermined operations, and various types of information such as values of measured blood pressure. The display unit 114 displays various types of information including the results of measurement of blood pressure. The operation unit 115 is operated to input various types of instructions for the measurement. The power supply unit 124 supplies power to the control unit 122 and the other functional blocks. The control unit 122 also functions as a blood pressure value calculating unit that calculates a value of blood pressure. The control unit 122 includes a central processing unit (CPU).

The pressure sensor 132 detects the pressure inside the air bladder 151 (hereinafter referred to as “cuff pressure” as appropriate), and outputs a signal corresponding to the detected pressure to the oscillation circuit 125. The pump 134 supplies air to the air bladder 151. The valve 135 opens and closes to maintain the pressure inside the air bladder 151 and to deflate the air bladder 151. The oscillation circuit 125 outputs, to the control unit 122, a signal having an oscillation frequency that depends on the value output from the pressure sensor 132. The pump drive circuit 126 controls the driving of the pump 134 in accordance with a control signal supplied from the control unit 122. The valve drive circuit 127 controls the opening and closing of the valve 135 in accordance with a control signal supplied from the control unit 122.

The operation unit 115 includes a power switch 115A, a measurement switch 115B, a stop switch 115C, and a user selection switch 115D. The power switch 115A is for switching between on and off of power supply to the blood pressure monitor 100. The measurement switch 115B is operated to cause the blood pressure monitor 100 to start the measurement of blood pressure. The stop switch 115C is operated to stop the ongoing operation of measuring blood pressure. The user selection switch 115D is for selecting a person to be measured by the blood pressure monitor 100.

The memory unit 123 stores therein the results of measurement, such as values of blood pressure and pulse rates, for each person that is measured. The stored results of measurement are displayed on the display unit 114 in a visible manner using numerical values, graphs, and the like. When the user selection switch 115D is operated on the blood pressure monitor 100, the results of measurement stored in the memory unit 123, such as values of blood pressure and pulse rates, are displayed for each person that is measured.

The memory unit 123 is constituted by a recording medium. The recording medium is, for example, a medium that stores therein programs in a non-volatile manner, such as a compact disc read-only memory (CD-ROM), a digital versatile disk read-only memory (DVD-ROM), a universal serial bus (USB) memory, a memory card, a flexible disk (FD), a hard disk, a magnetic tape, a cassette tape, a magnetic-optical (MO) disc, a MiniDisc (MD), an integrated circuit (IC) card (excluding memory cards), an optical card, a mask ROM, an EPROM, and an electronically erasable programmable read-only memory (EEPROM).

### 3. Configuration of Cuff

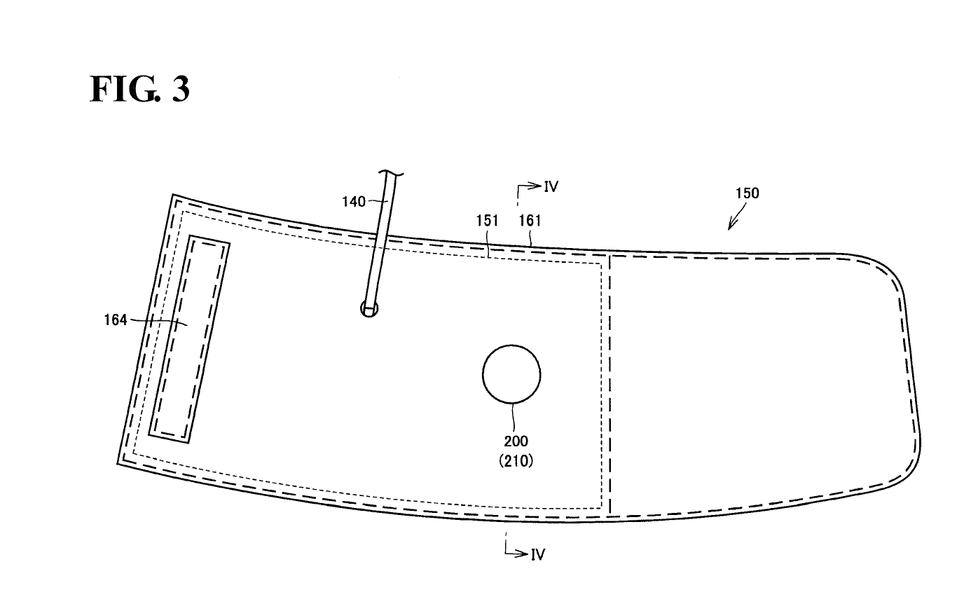

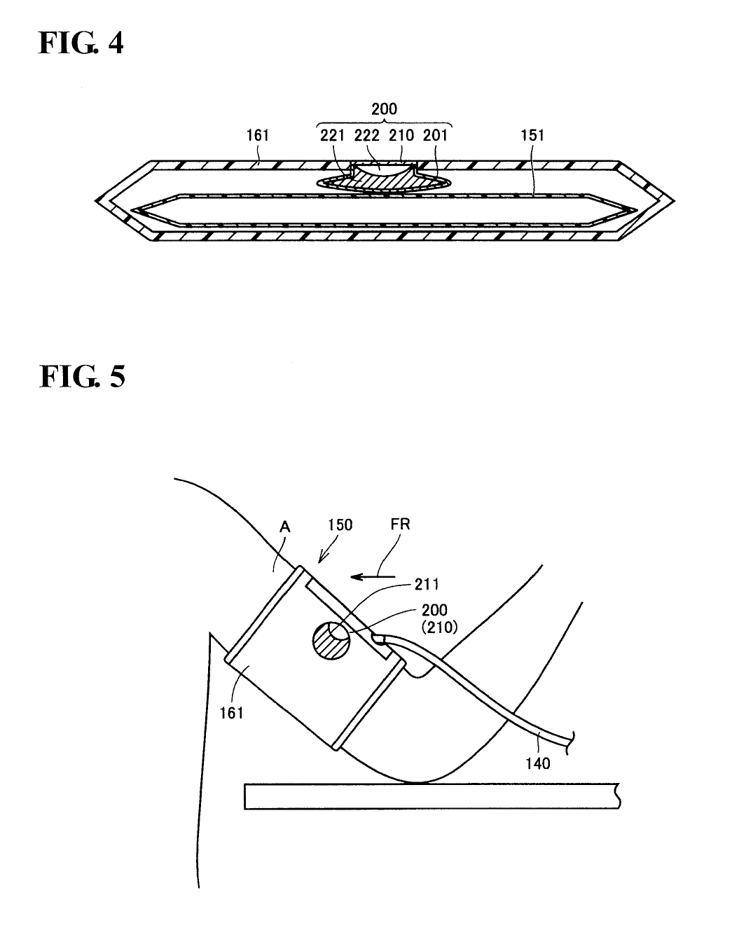

FIG. 3 shows the cuff 150 of the blood pressure monitor 100 in an opened state. FIG. 4 is a cross-sectional diagram of the cuff 150 taken along the IV-IV line shown in FIG. 3.

Referring to FIGS. 3 and 4, the cuff 150 includes the air bladder 151 connected to the air tube 140, and the bag-like cover body 161 containing the air bladder 151. As shown in FIG. 3, in the state where the cuff 150 is opened, the air bladder 151 has a substantially rectangular outer shape, and the bag-like cover body 161 has a substantially rectangular, belt-like outer shape to accommodate the air bladder 151.

The cuff 150, in a state of being wrapped around a measurement site (e.g. an upper arm), surrounds and covers the measurement site. A hook-and-loop fastener 164 is attached to the outer circumferential surface of the bag-like cover body 161 in the vicinity of one end of the bag-like cover body 161. In a state where the cuff 150 is attached to the upper arm, the hook-and-loop fastener 164 is fastened to a predetermined position on the outer circumferential surface of the bag-like cover body 161.

A wrapping strength indicating unit 200 is fitted into the bag-like cover body 161. The wrapping strength indicating unit 200 includes a window 210 and a fluid bag 201 attached to the window 210. A plurality of fluids (fluids 221 and 222) are enclosed in a space created by the window 210 and the fluid bag 201 of the wrapping strength indicating unit 200.

The fluid bag 201 is made of a material that easily deforms in shape by being pressed from the outside, such as a sheet-like synthetic resin. The window 210 is made of a transparent material such as transparent plastic, so that the state of the inside of the fluid bag 201 is visible from the outside. In the cuff 150, the window 210 is fitted into the surface of the bag-like cover body 161, and the fluid bag 201 is housed in the bag-like cover body 161. Inside the bag-like cover body 161, the fluid bag 201 is positioned between the bag-like cover body 161 and the air bladder 151 so as to contact the bag-like cover body 161 and the air bladder 151. In the present embodiment, the air bladder 151 constitutes a first fluid bag, and the fluid bag 201 constitutes a second fluid bag. Note that the fluid enclosed in the first fluid bag is not limited to air, but may be any fluid such as a certain gas.

The fluid 221 and the fluid 222 do not mix with each other and have different compressibilities. In the present embodiment, one of these two types of fluids (fluid 221) may be a liquid such as water, and the other (fluid 222) may be a gas such as air.

As has been described above, the cuff 150 is provided with an indicating member (wrapping strength indicating unit 200) that passes through an outer package (bag-like cover body 161) forming the outside of the cuff 150.

With the wrapping strength indicating unit 200, the enclosed fluids 221 and 222 (or at least one of them) are visible from the outside of the cuff 150 through the window 210. As mentioned above, the fluid bag 201 is made of a material that can deform by being pressed from the outside. Furthermore, when a certain force is applied from the outside of the fluid bag 201, both of (or at least one of) the fluids 221 and 222 change in volume. By visually checking such a change in the volume through the window 210, the person being measured can recognize what degree of force is being applied to the fluid bag 201. In the present embodiment, the window 210 constitutes a display unit of the indicating member (wrapping strength indicating unit 200).

When the cuff 150 of the blood pressure monitor 100 is wrapped around the measurement site, the fluid bag 201 is sandwiched between and pressed by the air bladder 151 and the bag-like cover body 161. As the wrapping strength of the cuff 150 increases, the force applied to the fluid bag 201 by the air bladder 151 and the bag-like cover body 161 increases. When the force applied to the fluid bag 201 changes, the volume of the fluids 221 and 222 (or at least one of them) in the fluid bag 201 changes. Therefore, with the use of the blood pressure monitor 100, it is possible to infer the strength at which the cuff 150 is wrapped around the measurement site by visually checking a change in the volume of the fluids 221 and 222 (or at least one of them) in the fluid bag 201 through the window 210.

In the present embodiment, the plurality of fluids (fluids 221 and 222) enclosed in the fluid bag 201 change in volume by different amounts when pressed from the outside with a certain force. Accordingly, when the force applied to the fluid bag 201 changes, the ratio between the volume of the fluid 221 and the volume of the fluid 222 in the fluid bag 201 changes. In the present embodiment, by visually checking a change in the volume ratio, the person being measured can infer the magnitude of the force applied to the fluid bag 201, namely the strength at which the cuff 150 is wrapped around the measurement site.

### 4. Confirmation of Wrapping Strength with Wrapping Strength Indicating Unit

FIG. 5 shows the state where the cuff 150 of the blood pressure monitor 100 has been wrapped around and fixed in place on an upper part of the (left) arm A of the person being measured, as one example of the state where the cuff 150 has been wrapped around and fixed in place on a measurement site. Note that arrow FR in FIG. 5 indicates a direction toward the person being measured from in front of the person. The cuff 150 of the blood pressure monitor 100 is intended to be attached to the measurement site such that the wrapping strength indicating unit 200 is positioned more toward the right side than toward the front, i.e. more toward the body of the person being measured than toward the front.

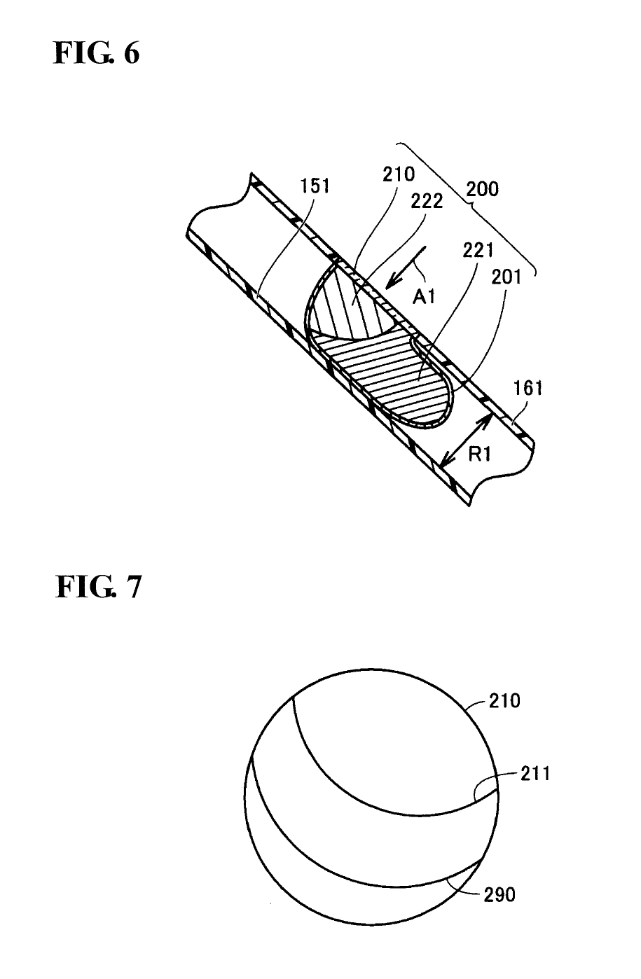

FIG. 6 shows a cross-sectional configuration of the cuff 150 in the state of FIG. 5 in the vicinity of the window 210 of the wrapping strength indicating unit.

Referring to FIG. 6, in the wrapping strength indicating unit 200 of the cuff 150, the window 210 is fixed in place by and housed in the bag-like cover body 161. When the bag-like cover body 161 of the cuff 150 is inclined with respect to the vertical direction as shown in FIG. 5, the fluid bag 201 hangs from the window 210 under its own weight, with a portion thereof fixed to the window 210 being located at the top. In this state, the fluid 222 is positioned above the fluid 221 inside the fluid bag 201. FIG. 7 shows the inside of the wrapping strength indicating unit 200 as viewed through the window 210 from the direction of arrow A1 when the fluid bag 201 is in the state shown in FIG. 6.

Referring to FIG. 7, a boundary between the fluids 221 and 222 can be seen as a boundary line 290 through the window 210.

The window 210 has an indicator 211 showing a position of the boundary line between the fluids 221 and 222 when the cuff 150 is wrapped around the measurement site at an appropriate strength. The indicator 211 is marked on the surface of the window 210 in advance by printing and the like.

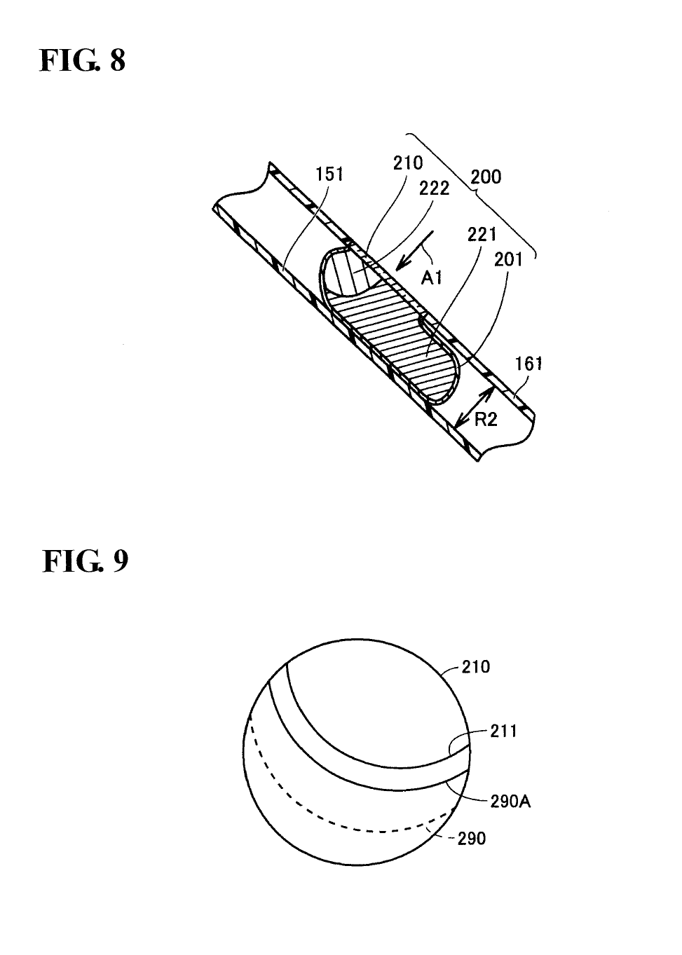

FIG. 8 is a cross-sectional diagram of the vicinity of the window 210 of the wrapping strength indicating unit 200 when the wrapping strength has been increased from the state shown in FIG. 6.

Note that R1 in FIGS. 6 and R2 in FIG. 8 indicate a distance between the air bladder 151 and the bag-like cover body 161 inside the bag-like cover body 161. Here, the following relationship holds: R1>R2. That is to say, in the state shown in FIG. 8, the distance between the air bladder 151 and the bag-like cover body 161 inside the bag-like cover body 161 is small as compared to the state shown in FIG. 6. This is based on the fact that the force applied to the bag-like cover body 161 from the measurement site has been increased. That is to say, the force with which the fluid bag 201 is squeezed by the bag-like cover body 161 and the air bladder 151 is larger in the state shown in FIG. 8 than in the state shown in FIG. 6.

The larger the force applied to the fluid bag 201 is, the more the fluids 221 and 222 are compressed. Note that the ratio between the volume of the fluid 221 and the volume of the fluid 222 in the fluid bag 201 changes from the state shown in FIG. 6 to the state shown in FIG. 8. More specifically, as the fluid 222 is compressed at a greater rate than the fluid 221 is, the proportion of the volume of the fluid 222 to the entirety of the fluid bag 201 is smaller in the state shown in FIG. 8 than in the state shown in FIG. 6.

FIG. 9 shows the inside of the fluid bag 201 as viewed through the window 210 from the direction of arrow A1 in the state shown in FIG. 8.

In FIG. 9, a boundary between the fluids 221 and 222 can be seen as a boundary line 290A. Note that FIG. 9 also shows the boundary line 290 of FIG. 7 for reference.

In the state shown in FIG. 8, the force applied to the fluid bag 201 is large, and therefore the fluid 222 is more condensed in the upper part of the fluid bag 201 and reduced in volume, as compared to the state shown in FIG. 6. Accordingly, the boundary between the fluids 221 and 222 is closer to the upper part of the window 210 in FIG. 9 than in FIG. 6.

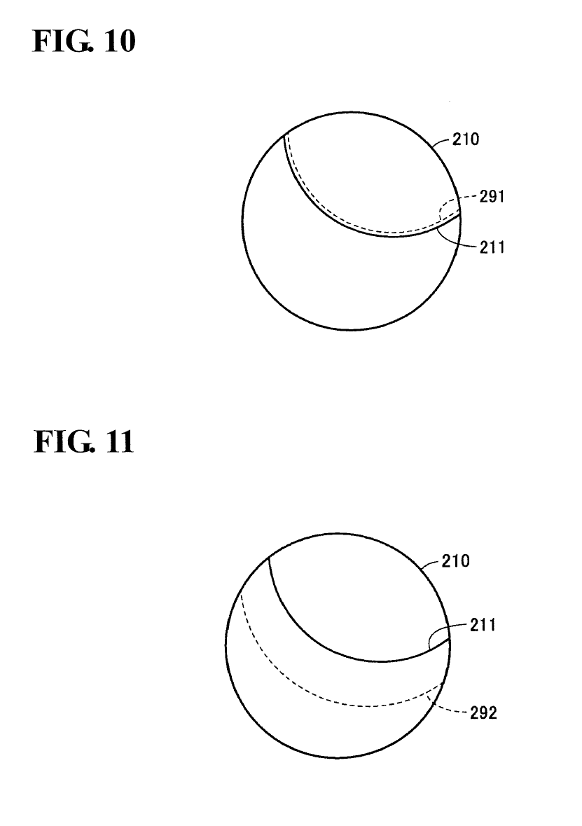

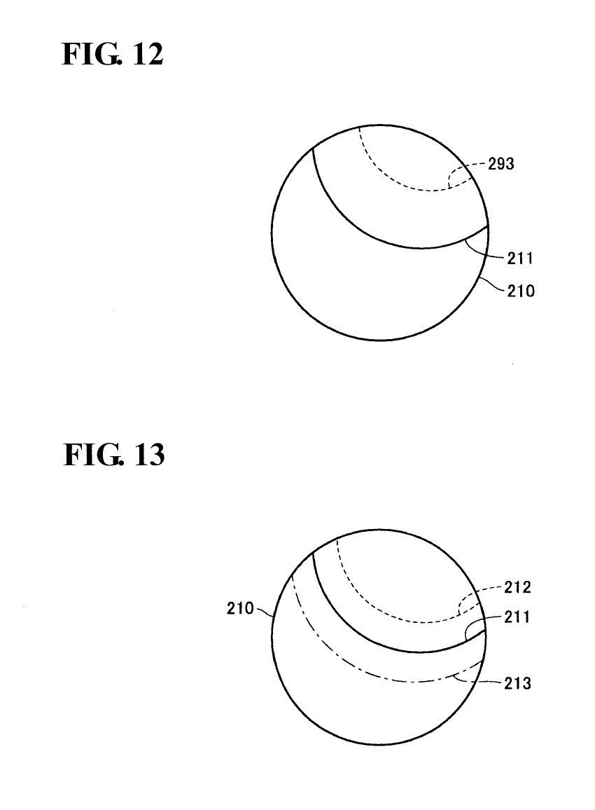

FIGS. 10 to 12 show how the position of the aforementioned boundary line changes in the window 210. In FIGS. 10 to 12, the aforementioned boundary line is given the reference signs 291 to 293. FIG. 10 shows the boundary line in the state where the cuff 150 is wrapped around the measurement site at an appropriate wrapping strength. FIG. 11 shows the boundary line in the state where the cuff 150 is wrapped around the measurement site at a wrapping strength that is too weak. FIG. 12 shows the boundary line in the state where the cuff 150 is wrapped around the measurement site at a wrapping strength that is too strong.

In FIG. 10, the boundary line 291 is positioned along the indicator 211. FIG. 11 shows the state where the boundary line 292 is positioned below the indicator 211. FIG. 12 shows the state where the boundary line 293 is positioned above the indicator 211.

As has been described above, with the blood pressure monitor 100, the position of the boundary line between the fluids 221 and 222 in the fluid bag 201, which is visible through the window 210, changes in accordance with the strength at which the cuff 150 is wrapped around the measurement site. In this way, by visually checking the position of the aforementioned boundary line in the window 210, the person being measured can determine whether or not the strength at which the cuff 150 is wrapped around the measurement site is appropriate.

The indicator 211 is marked on the window 210 in the present embodiment. In accordance with the positional relationship between the aforementioned boundary line and indicator 211, the person being measured by the blood pressure monitor 100 can determine whether or not the cuff 150 is wrapped around the measurement site at an appropriate strength.

As shown in FIGS. 10 to 12 and the like, the window 210 may be marked only with the indicator 211 showing where the boundary should be positioned when the cuff 150 is wrapped at an appropriate strength. Alternatively, as shown in FIG. 13, the window 210 may be marked with the indicator 211 showing an appropriate wrapping strength, the indicator 212 showing a wrapping strength that is too strong, and the indicator 213 showing a wrapping strength that is too weak.

### 5. Modes of Indicator of Wrapping Strength Indicating Unit

As shown in FIG. 10 and the like, the indicator 211 is provided on the window 210 in such a manner that it divides the surface of the window 210 into a plurality of areas to indicate the boundary line between the plurality of fluids in the fluid bag 201.

As can be understood from FIG. 5, in a state where it is intended that the cuff 150 is to be attached to the measurement site, the window 210 constitutes a surface that intersects a horizontal plane. Furthermore, in the wrapping strength indicating unit 200, the fluids 221 and 222 have different relative densities. Accordingly, in the blood pressure monitor 100 of the present embodiment, the centers of a plurality of shapes formed by the indicator 211 dividing the window 210 do not coincide with the center of the window 210, unlike a circular level gauge used in washing machines and the like, in which a circle having the same center as the circle forming the outside of the level gauge is formed.

### 6. Examples of Arrangement of Wrapping Strength Indicating Unit

### 6-1. Arrangement in Direction that Intersects Wrapping Direction

In the present embodiment, the blood pressure monitor 100 may be provided with a plurality of wrapping strength indicating units 200.

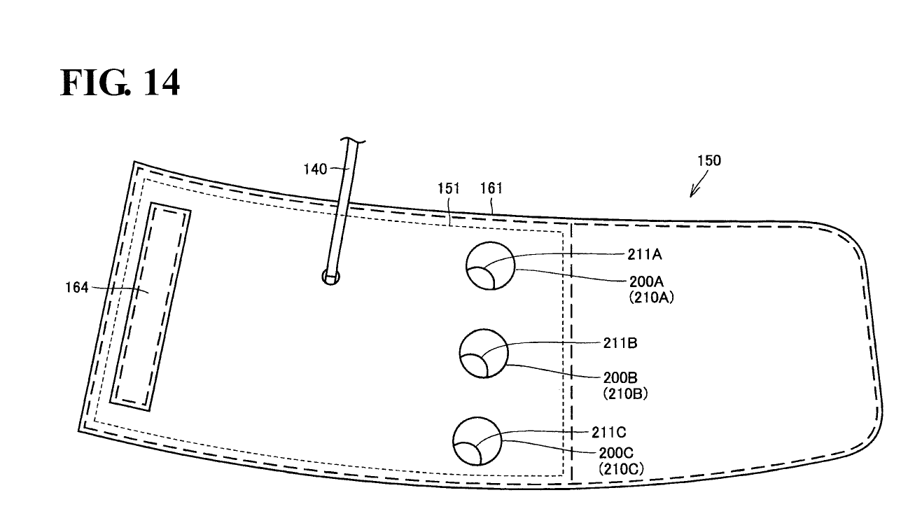

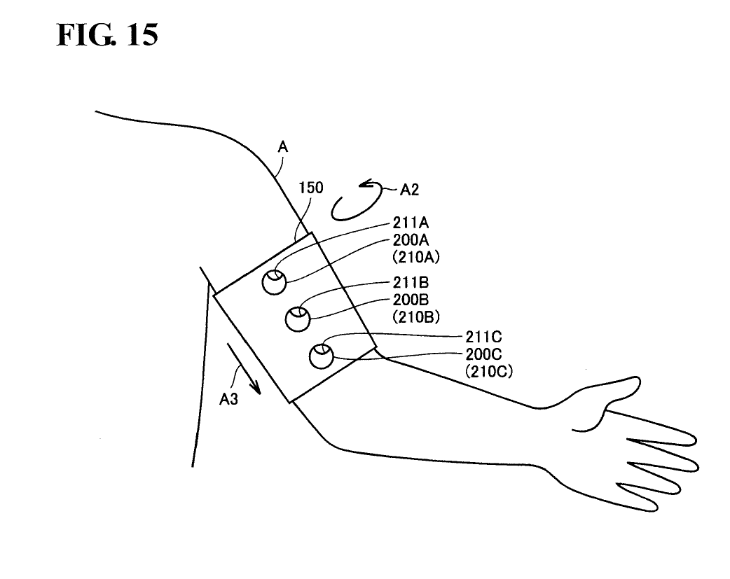

FIG. 14 shows a modification in which a plurality of wrapping strength indicating units 200A to 200C are arranged on the cuff 150 of the blood pressure monitor 100 in a direction that intersects the wrapping direction of the cuff 150. FIG. 15 shows the state where the cuff 150 of the modification shown in FIG. 14 is wrapped around the arm A of the person being measured. Note that in FIG. 15, arrow A2 indicates the wrapping direction of the cuff 150 around the measurement site (arm A), and arrow A3 indicates the direction that intersects the wrapping direction.

The wrapping strength indicating units 200A to 200C are configured in a similar manner to the wrapping strength indicating unit 200 described with reference to FIG. 4 and the like, and include windows 210A to 210C, respectively. The windows 210A to 210C are marked with indicators 211A to 211C (corresponding to the indicator 211), respectively.

As shown mainly in FIG. 15, the cuff 150 is intended to be wrapped around the measurement site such that the wrapping strength indicating units 200A to 200C are positioned slightly more toward the body of the person being measured than toward the front. When the cuff 150 is wrapped around the measurement site as in the state shown in FIG. 15, the indicators 211A to 211C of the windows 210A to 210C are located at the positions where it is assumed that the boundary between the fluids 221 and 222 in the fluid bag 201 will be located, namely, above the centers of the windows 210A to 210C and toward the right side with regard to the horizontal direction of FIG. 15. This makes it easy for the person being measured to visually check the windows 210A to 210C of the wrapping strength indicating units 200A to 200C when the cuff 150 is attached to the arm A.

When the plurality of wrapping strength indicating units 200A to 200C are arranged on the cuff 150 in the direction that intersects the wrapping direction of the cuff 150, the person being measured can visually check whether or not the wrapping strength of the cuff 150 is appropriate over a wider range in the direction that intersects the wrapping direction of the cuff 150.

### 6-2. Arrangement in Wrapping Direction

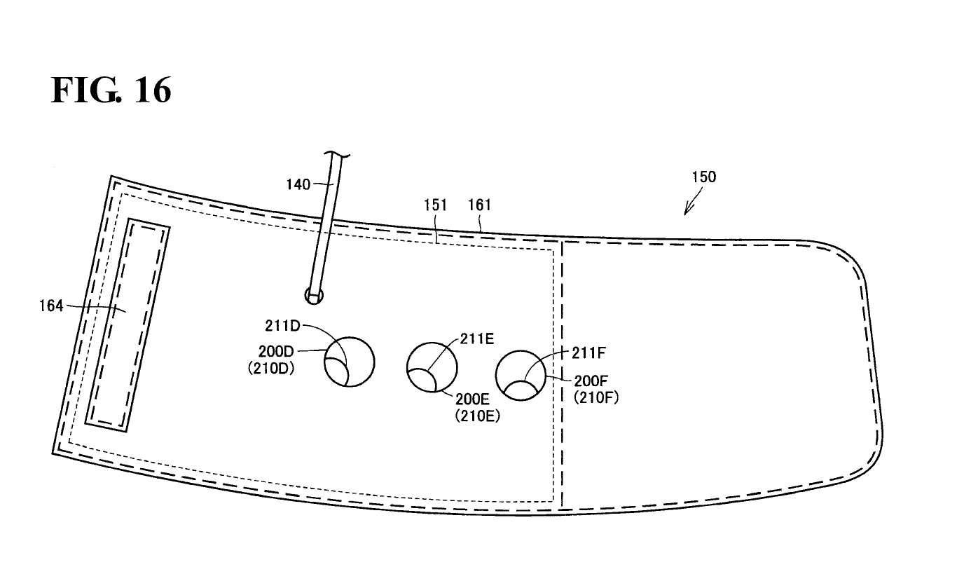

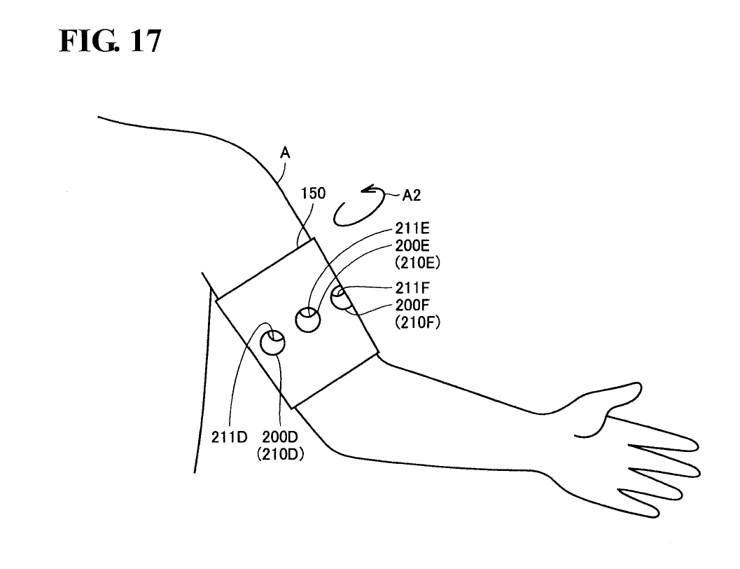

FIG. 16 shows a modification in which a plurality of wrapping strength indicating units 200D to 200F are arranged on the cuff 150 of the blood pressure monitor 100 in the wrapping direction of the cuff 150. FIG. 17 shows the state where the cuff 150 of the modification shown in FIG. 16 is wrapped around the arm A of the person being measured.

As shown mainly in FIG. 17, the cuff 150 of the present modification is intended to be wrapped around the arm A of the person being measured such that the wrapping strength indicating unit 200F at the farthest point is positioned in front of the person being measured, and the other wrapping strength indicating units 200D and 200E are positioned more toward the body of the person being measured than toward the front.

The wrapping strength indicating units 200D to 200F have windows 210D to 210F, respectively. The windows 210D to 210F are marked with indicators 211D to 211F, respectively.

Specifically, the windows 210D to 210F are marked with the indicators 211D to 211F in accordance with their positions when the cuff 150 is in a wrapped state.

As shown in FIG. 16, on the window 210F of the wrapping strength indicating unit 200F that is supposed to be positioned in front of the person being measured when the cuff 150 is attached to the arm A, the indicator 211F is marked substantially at the center of the window 210F in the horizontal direction.

When the cuff 150 is attached to the arm A, the wrapping strength indicating unit 200E is positioned closer to the body of the person being measured than the wrapping strength indicating unit 200F is in the wrapping direction indicated by arrow A2. As shown in FIG. 16, on the window 210E of the wrapping strength indicating unit 200E, the indicator 211E is marked at a position obtained by rotating the indicator 211F in a clockwise direction.

When the cuff 150 is attached to the arm A, the wrapping strength indicating unit 200D is positioned closer to the body of the person being measured than the wrapping strength indicating unit 200E is in the wrapping direction indicated by arrow A2. As shown in FIG. 16, on the window 210D of the wrapping strength indicating unit 200D, the indicator 211D is marked at a position obtained by rotating the indicator 211F in a clockwise direction (to a larger degree than the indicator 211E).

When the plurality of wrapping strength indicating units 200D to 200F are arranged in the wrapping direction of the cuff 150 (arrow A2) as in the present modification, the person being measured can visually check whether or not the wrapping strength is appropriate at a plurality of positions, i.e. over a wider range, in the wrapping direction of the cuff 150.

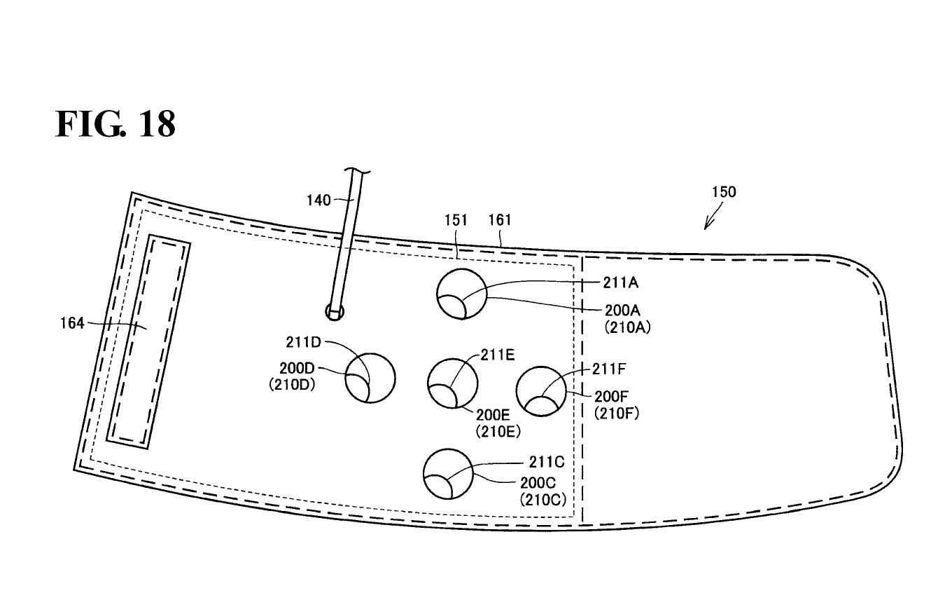

Alternatively, as shown in FIG. 18, the blood pressure monitor 100 may be provided with a plurality of wrapping strength indicating units 200D to 200F arranged in the wrapping direction of the cuff 150, and a plurality of wrapping strength indicating units 200A and 200C arranged in the direction that intersects the wrapping direction. With this arrangement, it is possible to yield the effects achieved by arranging a plurality of wrapping strength indicating units in the wrapping direction (the wrapping strength can be checked over a wide range in the wrapping direction), as well as the effects achieved by arranging a plurality of wrapping strength indicating units in the direction that intersects the wrapping direction (the wrapping strength can be checked over a wide range in the direction that intersects the wrapping direction).

### 7. Modifications of Material of Wrapping Strength Indicating Unit

In the present embodiment described above, the properties of the wrapping strength indicating unit 200, namely the mode of the plurality of fluids (fluids 221 and 222) enclosed in the space created by the fluid bag 201 and the window 210, change when the magnitude of the force applied to the fluid bag 201 by the bag-like cover body 161 and the air bladder 151 changes due to a change in the strength at which the cuff 150 is wrapped around the measurement site. It is possible to visually check whether or not the wrapping strength of the cuff 150 is appropriate by visually checking the change in the properties of the wrapping strength indicating unit 200 through the window 210.

The wrapping strength indicating unit 200 is not limited to the above-described configuration in which the plurality of fluids are enclosed, as long as it has a configuration in which its properties change in a visible manner due to a change in the force applied by the air bladder 151 and the bag-like cover body 161.

In order for the blood pressure monitor 100 to have a configuration in which the wrapping strength can be indirectly checked in a visible manner, the following materials may be used: a material with which the generated light energy increases in accordance with the magnitude of the mechanical energy applied from the outside (Xu, Chao-Nan. “Detection of Invisible Force Using Flash of Light.” The National Institute of Advanced Industrial Science and Technology (AIST) Kyushu. www.techno-qanda.net/Collection-10/Collection-15/Collection-102/Collection-470/Collection-2215/Document-16434/at_download/file); and a material that changes the color of emitted light in accordance with the magnitude of the force applied from the outside (Trafton, Anne. “MIT gel changes color on demand”. Oct. 21, 2007. http://web.mit.edu/newsoffice/2007/lightgels-1021.html).

When the latter material (that changes the color of emitted light in accordance with the magnitude of the force applied from the outside) is used for the wrapping strength indicating unit 200, according to one or more embodiments of the present invention, the cuff 150 is provided with a member indicating the color of light that it is assumed will be emitted by this material when the cuff 150 is wrapped at an appropriate wrapping strength as an indicator.

### 8. Processing for Measuring Blood Pressure

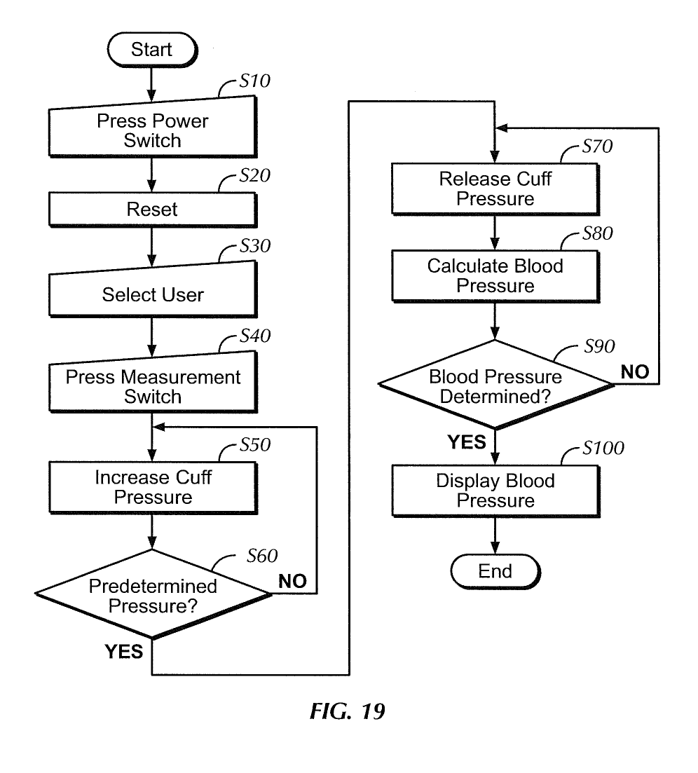

FIG. 19 is a flowchart of processing for measuring blood pressure executed by the blood pressure monitor 100. After the person being measured has wrapped the cuff 150 around the measurement site at an appropriate strength by visually checking the wrapping strength indicating unit 200 (wrapping strength indicating units 200A to 200F), the blood pressure monitor 100 executes the processing for measuring blood pressure upon operation of the power switch 115A or the like. Note that execution of this processing by the blood pressure monitor 100 is realized by the control unit 122 executing a program stored in the memory unit 123 (or a recording medium removable from the device main body 110).

Referring to FIG. 19, in the processing for measuring blood pressure, the control unit 122 first waits until the power switch 115A is operated in step S10. When the control unit 122 determines that the power switch 115 has been operated, it moves to the process of step S20.

In step S20, the control unit 122 resets the blood pressure monitor 100. Accordingly, the internal pressure of the air bladder 151 in the cuff 150 is reset.

Next, upon operation of the user selection switch 115D, the control unit 122 receives an input of information for selecting a user. When the control unit 122 determines that the information for selecting the user has been input, it moves to the process of step S40.

In step S40, the control unit 122 waits until the measurement switch 115B is operated. When the control unit 122 determines that the measurement switch 115B has been operated, it moves to the process of step S50.

In step S50, the control unit 122 increases the cuff pressure by causing the pump 134 to inflate the air bladder 151, and then moves to the process of step S60.

In step S60, the control unit 122 determines whether or not the cuff pressure has reached a predetermined pressure based on a signal output from the pressure sensor 132. When the control unit 122 determines that the cuff pressure has not reached the predetermined pressure yet, it returns to the process of step S50. On the other hand, when the control unit 122 determines that the cuff pressure has reached the predetermined pressure, it moves to the process of step S70.

In step S70, the control unit 122 gradually releases the cuff pressure by controlling the closed valve 135 to gradually open. Thereafter, in step S80, the control unit 122 calculates blood pressure (systolic blood pressure and diastolic blood pressure) in accordance with a predetermined procedure based on a pressure pulse wave signal superimposed on a signal detected by the pressure sensor 132 through the above deflation process. When it is determined in step S90 that the calculation of blood pressure has been completed (YES in step S90), the processing moves to step S100.

In step S100, the control unit 122 displays the values of blood pressure obtained in step S80 on the display unit 114 and ends the processing for measuring blood pressure.

Note that the obtained values of blood pressure are stored in the memory unit 123 in correspondence with the user selected in step S30.

Also, the control unit 122 deflates the air bladder 151 by controlling the valve 135 to fully open while (or after) displaying the values of blood pressure in step S 100.

### 9. Other Modifications

In the present embodiment described above, the blood pressure monitor 100 that measures blood pressure using the oscillometric method has been presented as one example of the blood pressure measurement device. However, a method for measuring blood pressure used by the blood pressure measurement device according to one or more embodiments of the present invention is not limited to this. One or more embodiments of the present invention may also be applied to a blood pressure measurement device that measures blood pressure using other methods (e.g. a microphone method).

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCE NUMERALS LIST

100 blood pressure monitor

110 device main body

114 display unit

115 operation unit

122 control unit

150 cuff

151 air bladder

161 bag-like cover body

200, 200A to 200F wrapping strength indicating unit

201 fluid bag

210 window

211, 211A to 211F indicator

221, 222 fluid

290, 290A, 291 to 293 boundary line

1. A blood pressure measurement device provided with a cuff that compresses a measurement site by being wrapped therearound, the blood pressure measurement device comprising:
an outer package that covers an outside of the cuff;
a first fluid bag provided inside the outer package; and
an indicating member provided in the cuff so as to contact the outer package and the first fluid bag,
wherein at least one property of the indicating member changes in a visible manner in accordance with a magnitude of a force with which the indicating member is pressed by the outer package and the first fluid bag.

2. The blood pressure measurement device according to claim 1,
wherein the indicating member (200) comprises a second fluid bag in which a plurality of fluids are enclosed, the plurality of fluids changing in volume by different amounts when pressed with a predetermined force.

3. The blood pressure measurement device according to claim 2,
wherein the indicating member further comprises a display unit that displays the change in the properties,
wherein the display unit is provided with an indicator that shows a magnitude of a force with which the second fluid bag is pressed by the outer package and the first fluid bag, and
wherein the indicator is marked on the display unit and has a shape whose center does not coincide with a center of the display unit.

4. The blood pressure measurement device according to claim 1,
wherein the indicating member is provided in plurality, and
wherein the plurality of indicating members are is arranged in a direction that intersects a wrapping direction in which the cuff is wrapped around the measurement site.

5. The blood pressure measurement device according to claim 1,
wherein the indicating member is provided in plurality, and
wherein the plurality of indicating members is arranged in a wrapping direction in which the cuff is wrapped around the measurement site.

6. The blood pressure measurement device according to claim 1, further comprising:
an indicator that shows a magnitude of a force with which the indicating member is pressed by the outer package and the first fluid bag.
